(19) United States
(12) Patent Application Publication
(10) Pub. No.: US 2025/0271754 A1
(43) Pub. Date: Aug. 28, 2025

(54) SEMICONDUCTOR PHOTORESIST COMPOSITIONS AND METHODS OF FORMING PATTERNS USING THE COMPOSITION

(71) Applicant: SAMSUNG SDI CO., LTD., Yongin-si (KR)

(72) Inventors: Taegeun SEONG, Suwon-si (KR); Seol Hee LIM, Suwon-si (KR); Sumin JANG, Suwon-si (KR); Seung-Wook SHIN, Suwon-si (KR); Yaeun SEO, Suwon-si (KR); Chungheon LEE, Suwon-si (KR)

(21) Appl. No.: 18/976,039

(22) Filed: Dec. 10, 2024

(30) Foreign Application Priority Data
Feb. 26, 2024 (KR) 10-2024-0027418

(51) Int. Cl. G03F 7/004; C07C 57/42; C07C 57/44; C07C 59/52; C07C 59/64; C07F 7/22; H01L 21/027

(52) U.S. Cl. CPC G03F 7/0042; C07C 57/42; C07C 57/44; C07C 59/52; C07C 59/64; C07F 7/2224; G03F 7/0045; H01L 21/0271

(57) ABSTRACT

A semiconductor photoresist composition and a method of forming patterns using the semiconductor photoresist composition are provided. The semiconductor photoresist composition includes a Sn-containing organometallic compound, a carboxylic acid compound including at least one aryl group and an unsaturated bond, and a solvent.

### CROSS-REFERENCE TO RELATED APPLICATION

The present application claims priority to and the benefit of Korean Patent Application No. 10-2024-0027418, filed on Feb. 26, 2024, in the Korean Intellectual Property Office, the entire content of which is incorporated herein by reference.

### BACKGROUND

### 1. Field

One or more embodiments of the present disclosure relate to semiconductor photoresist compositions and methods of forming patterns using the semiconductor photoresist composition.

### 2. Description of the Related Art

EUV (extreme ultraviolet) lithography has drawn much attention as one essential technology for manufacturing a next generation semiconductor device. That is, for the production of next-generation semiconductor devices, EUV lithography has garnered significant attention as a crucial technology. The EUV lithography is a pattern-forming technology using an EUV ray having a wavelength of 13.5 nm as an exposure light source. According to the EUV lithography, an extremely fine pattern (e.g., less than or equal to 20 nm) may be formed in an exposure process during a manufacture of a semiconductor device (e.g., a semiconductor chip).

The extreme ultraviolet (EUV) lithography is realized through development of compatible photoresists which may be performed at a spatial resolution of less than or equal to 16 nm. Currently, efforts to alleviate insufficient specifications of chemically amplified (CA) photoresists such as a resolution, a photospeed, and feature roughness (or also referred to as a line edge roughness or LER) for the next generation device are being made.

An intrinsic image blurring due to an acid catalyzed reaction in these polymer-type or kind photoresists limits a resolution in small feature sizes, which has existed in electron beam (e-beam) lithography for a long time. The chemically amplified (CA) photoresists are designed for high sensitivity, but because their typical elemental makeups reduce light absorbance of the photoresists at a wavelength of 13.5 nm, it may decrease their sensitivity, and the chemically amplified (CA) photoresists may partially have more difficulties under an EUV exposure.

In addition, the CA photoresists may have difficulties in the small feature sizes due to roughness issues, and line edge roughness (LER) of the CA photoresists experimentally may be increased, as a photospeed may be decreased partially due to an essence of acid catalyst processes. Accordingly, a novel high-performance photoresist is desired or required in a semiconductor industry because of these defects and problems of the CA photoresists.

In order to overcome the aforementioned drawbacks of the chemically amplified (CA) organic photosensitive composition, an inorganic photosensitive composition has been researched. The inorganic photosensitive composition has been mainly or predominantly used for negative tone patterning which has resistance against removal by a developer composition due to chemical modification through non-chemical amplification mechanism. The inorganic composition contains an inorganic element that has a higher EUV absorption rate than hydrocarbon, and thus may secure sensitivity through the nonchemical amplification mechanism and may be less sensitive about a stochastic effect and thus may have low line edge roughness and small number of defects.

Inorganic photoresists based on peroxopolyacids of tungsten mixed with tungsten, niobium, titanium, and/or tantalum have been reported as radiation sensitive materials for patterning.

These materials are effective for patterning large pitches for bilayer configuration as far ultraviolet (deep UV), X-ray, and electron beam sources. When cationic hafnium metal oxide sulfate (HfSOx) materials along with a peroxo complexing agent was used to image a 15 nm half-pitch (HP) through projection EUV exposure, improved performance has been obtained. This system exhibits high performance of a non-CA photoresist and has a practicable photospeed near to a requirement for an EUV photoresist. However, the hafnium metal oxide sulfate material having the peroxo complexing agent has some practical drawbacks. First, these materials are coated in a mixture of corrosive sulfuric acid/hydrogen peroxide and have insufficient shelf-life stability. Second, a structural change of the materials for performance improvement as a composite mixture is challenging. Third, development should be performed in a TMAH (tetramethylammonium hydroxide) solution at an extremely high concentration of 25 wt % and/or the like.

To address these issues, research has been focused on developing molecules that contain tin, which have excellent or suitable absorption of extreme ultraviolet rays. As for an organotin polymer among the molecules containing tin, alkyl ligands are dissociated by light absorption or secondary electrons produced. The dissociated alkyl ligands are then crosslinked with adjacent chains through oxo bonds and thus, enable the negative tone patterning which may not be removed by an organic developer. Although this organotin polymer exhibits greatly improved sensitivity as well as maintains a resolution and line edge roughness, the patterning characteristics need to be further improved for commercial availability.

### SUMMARY

One or more aspects of embodiments of the present disclosure are directed toward a semiconductor photoresist composition having excellent or suitable sensitivity, line edge roughness (LER), and resolution characteristics.

One or more aspects of embodiments of the present disclosure are directed toward a method of forming patterns using the semiconductor photoresist composition.

Additional aspects will be set forth in part in the description which follows and, in part, will be apparent from the description, or may be learned by practice of the presented embodiments of the disclosure.

According to one or more embodiments, a semiconductor photoresist composition includes a Sn-containing organometallic compound, a carboxylic acid compound including at least one aryl group and an unsaturated bond, and a solvent.

According to one or more embodiments, a method of forming patterns includes forming an etching-objective layer (e.g., etching-target layer) on a substrate, coating the semiconductor photoresist composition on the etching-objective layer to form a photoresist layer, patterning the photoresist layer to form a photoresist pattern, and etching the etching-objective layer using the photoresist pattern as an etching mask.

The semiconductor photoresist composition according to one or more embodiments enables implementation of excellent or suitable sensitivity and excellent or suitable LER characteristics.

### BRIEF DESCRIPTION OF DRAWINGS

The above and other aspects, features, and advantages of certain embodiments of the disclosure will be more apparent from the following description taken in conjunction with the accompanying drawings, in which:

FIGS. 1A-1E are cross-sectional views for illustrating a method of forming patterns using a semiconductor photoresist composition according to one or more embodiments of the present disclosure.

### DETAILED DESCRIPTION

The present disclosure may be modified in many alternate forms, and thus specific embodiments will be exemplified in the drawing and described in more detail. It should be understood, however, that it is not intended to limit the present disclosure to the particular forms disclosed, but rather, is intended to cover all modifications, equivalents, and alternatives falling within the spirit and scope of the present disclosure.

Hereinafter, referring to the drawings, example embodiments are described in more detail. In the following description of the present disclosure, the well-established functions or constructions will not be described in order to make the present disclosure concise.

In order to clearly illustrate the present disclosure, the unessential description and relationships are omitted, and throughout the disclosure, the same or similar configuration elements are designated by the same reference numerals. Also, because the size and thickness of each configuration shown in the drawing are shown for better understanding and ease of description, the present disclosure is not necessarily limited thereto.

In the drawings, the thickness of layers, films, panels, regions, and/or the like, may be enlarged for clarity. In the drawings, the thickness of a part of layers or regions, and/or the like, may be exaggerated for clarity. It will be understood that if (e.g., when) an element such as a layer, film, region, or substrate is referred to as being “on” another element, it may be directly on the other element or intervening elements may also be present therebetween. In contrast, when an element is referred to as being “directly on” another element, there are no intervening elements present.

As used herein, “substituted” refers to replacement of a hydrogen by deuterium, a halogen, a hydroxyl group, a carboxyl group, a thiol group, a cyano group, a nitro group, —NRR′ (wherein, R and R′ may each independently be hydrogen, a substituted or unsubstituted C1 to C30 saturated or unsaturated aliphatic hydrocarbon group, a substituted or unsubstituted C3 to C30 saturated or unsaturated alicyclic hydrocarbon group, or a substituted or unsubstituted C6 to C30 aromatic hydrocarbon group), —SiRR′R″ (wherein, R, R′, and R″ may each independently be hydrogen, a substituted or unsubstituted C1 to C30 saturated or unsaturated aliphatic hydrocarbon group, a substituted or unsubstituted C3 to C30 saturated or unsaturated alicyclic hydrocarbon group, or a substituted or unsubstituted C6 to C30 aromatic hydrocarbon group), a C1 to C30 alkyl group, a C1 to C10 haloalkyl group, a C1 to C10 alkylsilyl group, a C3 to C30 cycloalkyl group, a C6 to C30 aryl group, a C1 to C20 alkoxy group, a C1 to C20 sulfide group, and/or a (e.g., any suitable) combination thereof. “Unsubstituted” refers to non-replacement of a hydrogen by another substituent and remaining of the hydrogen.

As used herein, if (e.g., when) a definition is not otherwise provided, the term “alkyl group” refers to a linear or branched aliphatic hydrocarbon group. The alkyl group may be a “saturated alkyl group” without any double bond or triple bond.

The alkyl group may be a C1 to C8 alkyl group. For example, the alkyl group may be a C1 to C7 alkyl group, a C1 to C6 alkyl group, or a C1 to C5 alkyl group. For example, the C1 to C5 alkyl group may be a methyl group, an ethyl group, a propyl group, an isopropyl group, an n-butyl group, an isobutyl group, a sec-butyl group, a tert-butyl group, or a 2,2-dimethylpropyl group.

As used herein, if (e.g., when) a definition is not otherwise provided, the term “cycloalkyl group” refers to a monovalent cyclic aliphatic hydrocarbon group.

The cycloalkyl group may be a C3 to C8 cycloalkyl group, a C3 to C7 cycloalkyl group, a C3 to C6 cycloalkyl group, a C3 to C5 cycloalkyl group, or a C3 to C4 cycloalkyl group. For example, the cycloalkyl group may be a cyclopropyl group, a cyclobutyl group, a cyclopentyl group, or a cyclohexyl group, but the present disclosure is not limited thereto.

As used herein, the term “aliphatic unsaturated organic group” refers to a hydrocarbon group including a bond in which the bond between the carbon and carbon atom in the group is a double bond, a triple bond, or a (e.g., any suitable) combination thereof.

The aliphatic unsaturated organic group may be a C2 to C8 aliphatic unsaturated organic group. For example, the aliphatic unsaturated organic group may be a C2 to C7 aliphatic unsaturated organic group, a C2 to C6 aliphatic unsaturated organic group, a C2 to C5 aliphatic unsaturated organic group, or a C2 to C4 aliphatic unsaturated organic group. For example, the C2 to C4 aliphatic unsaturated organic group may be a vinyl group, an ethynyl group, an allyl group, a 1-propenyl group, a 1-methyl-1-propenyl group, a 2-propenyl group, a 2-methyl-2-propenyl group, a 1-propynyl group, a 1-methyl-1-propynyl group, a 2-propynyl group, a 2-methyl-2-propynyl group, a 1-butenyl group, a 2-butenyl group, a 3-butenyl group, a 1-butynyl group, a 2-butynyl group, or a 3-butynyl group.

As used herein, the term “aryl group” refers to a substituent in which all atoms in the cyclic substituent have a p-orbital and these p-orbitals are conjugated and may include a monocyclic or fused ring polycyclic functional group (i.e., rings sharing adjacent pairs of carbon atoms).

As used herein, the term “heteroaryl group” may refer to an aryl group including at least one heteroatom selected from among N, O, S, P, and Si. Two or more heteroaryl groups may be linked by a sigma bond directly, or if (e.g., when) the heteroaryl group includes two or more rings, the two or more rings may be fused. When the heteroaryl group is a fused ring, each ring may independently include one to three heteroatoms.

As used herein, unless otherwise defined, the term “alkenyl group” refers to an aliphatic unsaturated alkenyl group including at least one carbon-carbon double bond as a linear or branched aliphatic hydrocarbon group.

As used herein, unless otherwise defined, the term “alkynyl group” refers to an aliphatic unsaturated alkynyl group including at least one carbon-carbon triple bond as a linear or branched aliphatic hydrocarbon group.

Hereinafter, a semiconductor photoresist composition according to one or more embodiments will be described in more detail.

The semiconductor photoresist composition according to one or more embodiments includes a Sn-containing organometallic compound, a carboxylic acid compound including at least one aryl group and an unsaturated bond, and a solvent. For example, the unsaturated bond may not be included in the at least one aryl group.

For example, in one or more embodiments, the carboxylic acid compound may be a compound represented by Chemical Formula 1 or Chemical Formula 2.

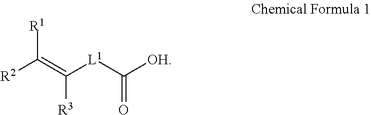

In Chemical Formula 1,
R¹ to R³ may each independently be hydrogen, a halogen, a hydroxyl group, a substituted or unsubstituted C1 to C20 alkyl group, a substituted or unsubstituted C6 to C30 aryl group, or a (e.g., any suitable) combination thereof,
at least one selected from among R¹ to R³ may be a substituted or unsubstituted C6 to C30 aryl group, and
L¹ is a single bond or a substituted or unsubstituted C1 to C10 alkylene group;

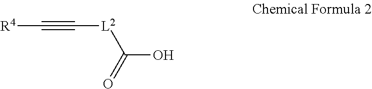

wherein, in Chemical Formula 2,
R⁴ may be a substituted or unsubstituted C6 to C30 aryl group, and
L² is a single bond or a substituted or unsubstituted C1 to C10 alkylene group.

The semiconductor photoresist composition may improve sensitivity and LER and achieve excellent or suitable resolution by applying a carboxylic acid compound including both (e.g., simultaneously) an aryl group and an unsaturated bond.

For example, in one or more embodiments, at least one of R¹ or R² of Chemical Formula 1 or R³ may each independently be a substituted or unsubstituted C6 to C30 aryl group.

In one or more embodiments, at least one selected from among R¹ and R², or R³ in Chemical Formula 1 and R⁴ in Chemical Formula 2 may each independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, or a substituted or unsubstituted triphenylene group.

In one or more embodiments, the carboxylic acid compound may be any one selected from among compounds listed in Group 1.

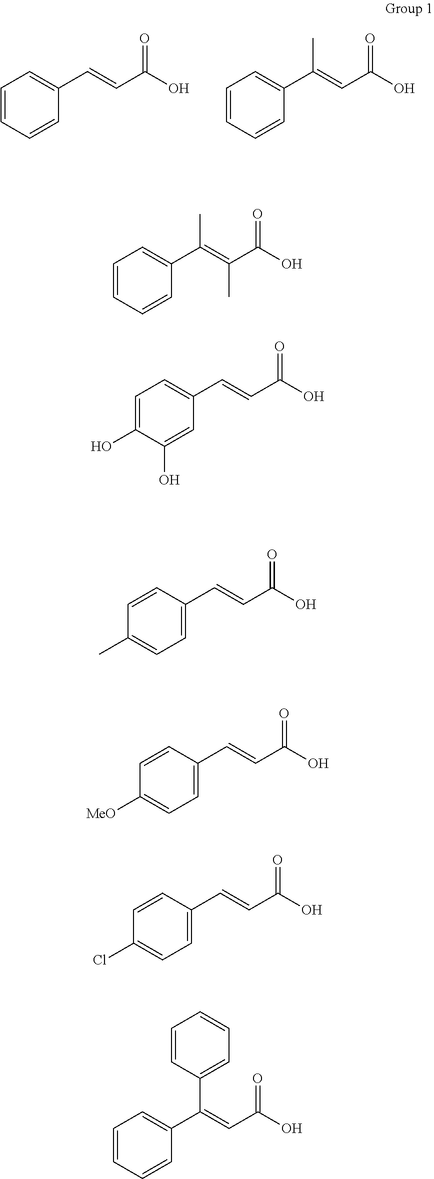

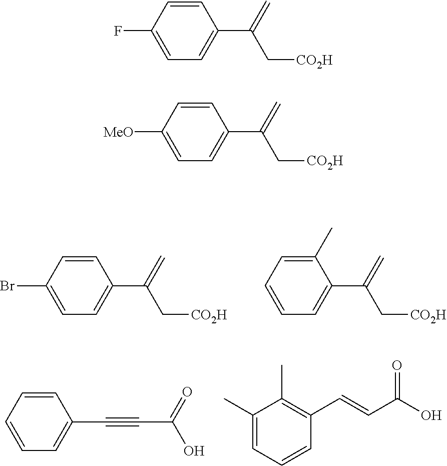

The carboxylic acid compound may be included in an amount of about 0.001 to about 10 wt % based on 100 wt % of a total weight of the semiconductor photoresist composition.

In one or more embodiments, the carboxylic acid compound may be included in an amount of about 0.01 to about 10 wt %, about 0.01 to about 5 wt %, about 0.05 to about 5 wt % or about 0.1 to about 5 wt % based on 100 wt % of the total weight of the semiconductor photoresist composition.

The Sn-containing organometallic compound may be included in an amount of about 0.5 wt % to about 30 wt % based on 100 wt % of the total weight of the semiconductor photoresist composition.

The semiconductor photoresist composition according to one or more embodiments may improve the sensitivity of the photoresist by including the Sn-containing organometallic compound and the carboxylic acid compound within the above content (e.g., amount) range.

The semiconductor photoresist composition according to one or more embodiments may include the Sn-containing organometallic compound and the carboxylic acid compound in a weight ratio of about 99:1 to about 70:30. In one or more embodiments, the semiconductor photoresist composition may include the Sn-containing organometallic compound and the carboxylic acid compound in a weight ratio of about 99:1 to about 80:20.

If the weight ratio of the Sn-containing organometallic compound and the carboxylic acid compound satisfies the above range, a semiconductor photoresist composition having excellent or suitable sensitivity may be provided.

The Sn-containing organometallic compound may include at least one of an organic oxy group or an organic carbonyloxy group.

In one or more embodiments, the Sn-containing organometallic compound may be represented by Chemical Formula 3.

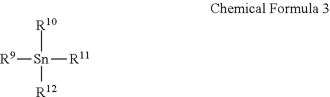

In Chemical Formula 3,
R⁹ may be selected from among a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, and a substituted or unsubstituted C7 to C30 arylalkyl group,
R¹⁰ to R¹² may each independently be a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, a substituted or unsubstituted C7 to C30 arylalkyl group, an alkoxy or aryloxy group (—OR^{a}, wherein R^{a} may be a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a (e.g., any suitable) combination thereof), a carboxyl group (—O(C═O)R^{b}, wherein R^{b} may be hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a (e.g., any suitable) combination thereof), an alkylamido or dialkylamido group (—NR^{c}R^{d}, wherein R^{c} and R^{d} may each independently be hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a (e.g., any suitable) combination thereof), an amidato group (—NRₑ (C═OR^{f}), wherein R^{e} and R^{f} may each independently be hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a (e.g., any suitable) combination thereof), an amidinato group (—NR^{g}C(NR^{h})Rⁱ, wherein R^{g}, R^{h}, and Rⁱ may each independently be hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a (e.g., any suitable) combination thereof), an alkylthio or arylthiol group (—SRⁱ, wherein Rⁱ may be a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a (e.g., any suitable) combination thereof), or a thiocarboxyl group (—S(C═O)R^{k}, wherein R^{k} may be hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a (e.g., any suitable) combination thereof), and
at least one selected from among R¹⁰ to R¹² may be an alkoxy or aryloxy group (—OR^{a}, wherein R^{a} may be a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a (e.g., any suitable) combination thereof), a carboxyl group (—O(C═O)R^{b}, wherein R^{b} may be hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a (e.g., any suitable) combination thereof), an alkylamido or dialkylamido group (—NR^{c}R^{d}, wherein R^{c} and R^{d} may each independently be hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a (e.g., any suitable) combination thereof), an amidato group (—NR^{e}(C═OR^{f}), wherein R^{e} and R^{f} may each independently be hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a (e.g., any suitable) combination thereof), an amidinato group (—NR^{g}C(NR^{h})Rⁱ, wherein R^{g}, R^{h}, and Rⁱ may each independently be hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a (e.g., any suitable) combination thereof), an alkylthio or arylthiol group (—SRⁱ, wherein Rⁱ may be a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a (e.g., any suitable) combination thereof), or a thiocarboxyl group (—S(C═O)R^{k}, wherein R^{k} may be hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a (e.g., any suitable) combination thereof).

In one or more embodiments, at least one selected from among R¹⁰ to R¹² may be selected from among an alkoxy or aryloxy group (—OR^{a}, wherein R^{a} may be a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a (e.g., any suitable) combination thereof), and a carboxyl group (—O(C═O)R^{b}, wherein R^{b} may be hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a (e.g., any suitable) combination thereof).

In one or more embodiments, the compound represented by Chemical Formula 3 includes —OR^{a} or —OC(═O)R^{b} as a ligand, so that a pattern formed using a semiconductor photoresist composition including the compound may exhibit excellent or suitable limiting resolution.

Additionally, the —OR^{a} or —OC(═O)R^{b} ligand may determine the solubility of the compound represented by Chemical Formula 3 in a solvent.

In one or more embodiments, R⁹ may be a substituted or unsubstituted C1 to C8 alkyl group, a substituted or unsubstituted C3 to C8 cycloalkyl group, a substituted or unsubstituted C2 to C8 aliphatic unsaturated organic group including one or more double bonds or triple bonds, a substituted or unsubstituted C6 to C20 aryl group, a substituted or unsubstituted C4 to C20 heteroaryl group, a carbonyl group, an ethoxy group, a propoxy group, or a (e.g., any suitable) combination thereof,
R^{a} may be a substituted or unsubstituted C1 to C8 alkyl group, a substituted or unsubstituted C3 to C8 cycloalkyl group, a substituted or unsubstituted C2 to C8 alkenyl group, a substituted or unsubstituted C2 to C8 alkynyl group, a substituted or unsubstituted C6 to C20 aryl group, or a (e.g., any suitable) combination thereof, and
R^{b} may be hydrogen, a substituted or unsubstituted C1 to C8 alkyl group, a substituted or unsubstituted C3 to C8 cycloalkyl group, a substituted or unsubstituted C2 to C8 alkenyl group, a substituted or unsubstituted C2 to C8 alkynyl group, a substituted or unsubstituted C6 to C20 aryl group, or a (e.g., any suitable) combination thereof.

In one or more embodiments, R⁹ may be a methyl group, an ethyl group, a propyl group, a butyl group, an isopropyl group, a tert-butyl group, a 2,2-dimethylpropyl group, a cyclopropyl group, a cyclobutyl group, a cyclopentyl group, a cyclohexyl group, an ethenyl group, a propenyl group, a butenyl group, an ethynyl group, a propynyl group, a butynyl group, a phenyl group, a tolyl group, a xylene group, a benzyl group, a formyl group, an acetyl group, a propanoyl group, a butanoyl group, a pentanoyl group, an ethoxy group, a propoxy group, or a (e.g., any suitable) combination thereof,
R^{a} may be an ethyl group, a propyl group, a butyl group, an isopropyl group, a tert-butyl group, a 2,2-dimethylpropyl group, a cyclopropyl group, a cyclobutyl group, a cyclopentyl group, a cyclohexyl group, an ethenyl group, a propenyl group, a butenyl group, an ethynyl group, a propynyl group, a butynyl group, a phenyl group, a tolyl group, a xylene group, a benzyl group, or a (e.g., any suitable) combination thereof, and
R^{b} may be hydrogen, an ethyl group, a propyl group, a butyl group, an isopropyl group, a tert-butyl group, a 2,2-dimethylpropyl group, a cyclopropyl group, a cyclobutyl group, a cyclopentyl group, a cyclohexyl group, an ethenyl group, a propenyl group, a butenyl group, an ethynyl group, a propynyl group, a butynyl group, a phenyl group, a tolyl group, a xylene group, a benzyl group, or a (e.g., any suitable) combination thereof.

In one or more embodiments, the Sn-containing organometallic compound may be represented by Chemical Formula 4 or Chemical Formula 5.

R¹³_{z}SnO_{(2-(z/2)-(x/2))}(OH)ₓ Chemical Formula 4

In Chemical Formula 4,

R¹³ may be a C1 to C31 hydrocarbyl group, 0<z≤2, and 0<(z+x)≤4;

R¹⁴ₐ₁Sn_{b1}X_{c1}Y_{d1} Chemical Formula 5
wherein, in Chemical Formula 5,
R¹⁴ may be a substituted or unsubstituted C1 to C20 alkyl group, a substituted or unsubstituted C3 to C20 cycloalkyl group, a substituted or unsubstituted C2 to C20 aliphatic unsaturated organic group including one or more double bonds or triple bonds, a substituted or unsubstituted C6 to C30 aryl group, a substituted or unsubstituted C4 to C30 heteroaryl group, a carbonyl group, an ethylene oxide group, a propylene oxide group, or a (e.g., any suitable) combination thereof,
X may be sulfur(S), selenium (Se), or tellurium (Te),
Y may be —OR^{l} or —OC(═O)R^{m},
wherein R^{l} may be a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a (e.g., any suitable) combination thereof,
R^{m} may be hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a (e.g., any suitable) combination thereof, and
a1, b1, c1, and d1 may each independently be an integer of 1 to 20.

The solvent of the semiconductor photoresist composition according to one or more embodiments may be an organic solvent, and may be, for example, selected from aromatic compounds (e.g., xylene, toluene, and/or the like), alcohols (e.g., 4-methyl-2-pentanol, 4-methyl-2-propanol, 1-butanol, methanol, isopropyl alcohol, 1-propanol), ethers (e.g., anisole, tetrahydrofuran), esters (n-butyl acetate, propylene glycol monomethyl ether acetate, ethyl acetate, ethyl lactate), ketones (e.g., methyl ethyl ketone, 2-heptanone), and/or a (e.g., any suitable) mixture thereof, but the present disclosure is not limited thereto.

The semiconductor photoresist composition according to one or more embodiments may further include a resin in addition to the aforementioned Sn-containing organometallic compound, carboxylic acid compound, and solvent.

The resin may be a phenol-based resin including at least one aromatic moiety selected from moieties of Group 2.

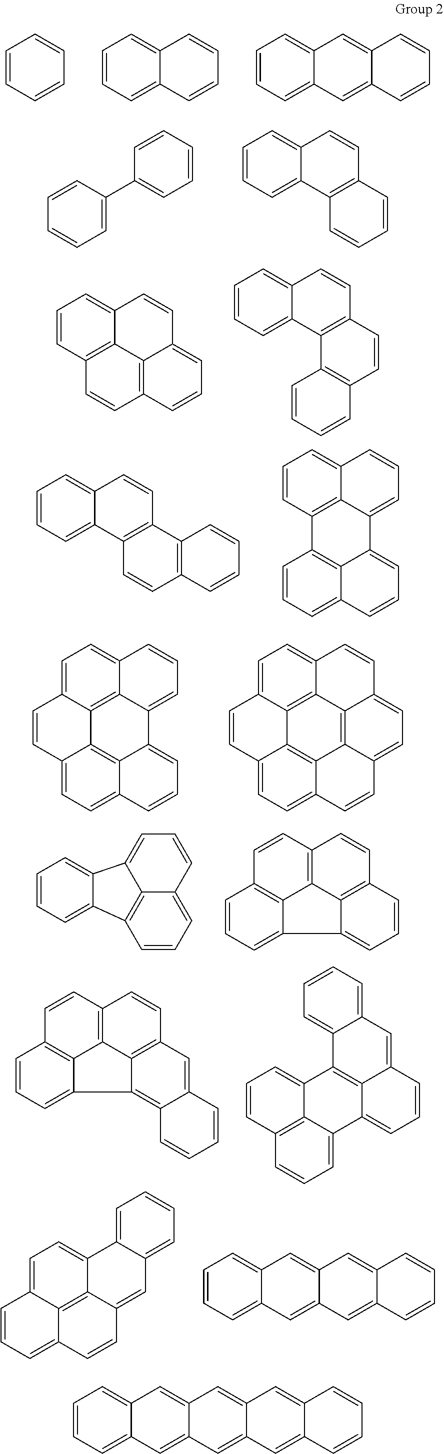

The resin may have a weight average molecular weight of about 500 to about 20,000.

In one or more embodiments, the resin may be included in an amount of about 0.1 wt % to about 50 wt % based on 100 wt % of a total amount of the semiconductor photoresist composition.

When the resin is included in the above content (e.g., amount) range, it may have excellent or suitable etch resistance and heat resistance.

In addition, the semiconductor photoresist composition according to one or more embodiments may include (e.g., consist of) the aforementioned Sn-containing organometallic compound, carboxylic acid compound, solvent, and resin.

In the present disclosure, the semiconductor photoresist composition according to one or more embodiments may further include one or more additives as needed. Non-limiting examples of the additives may be a surfactant, a crosslinking agent, a leveling agent, an organic acid, a quencher, and/or a (e.g., any suitable) combination thereof.

The surfactant may include, for example, an alkyl benzene sulfonate salt, an alkyl pyridinium salt, polyethylene glycol, a quaternary ammonium salt, and/or a (e.g., any suitable) combination thereof, but embodiments of the present disclosure are not limited thereto.

The crosslinking agent may be, for example, a melamine-based crosslinking agent, a substituted urea-based crosslinking agent, an acryl-based crosslinking agent, an epoxy-based crosslinking agent, or a polymer-based crosslinking agent, but embodiments of the present disclosure are not limited thereto. It may be a crosslinking agent having at least two crosslinking forming substituents, for example, a compound such as methoxymethylated glycoluril, butoxymethylated glycoluril, methoxymethylated melamine, butoxymethylated melamine, methoxymethylated benzoguanamine, butoxymethylated benzoguanamine, 4-hydroxybutyl acrylate, acrylic acid, urethane acrylate, acryl methacrylate, 1,4-butanediol diglycidyl ether, glycidol, diglycidyl 1,2-cyclohexane dicarboxylate, trimethylpropane triglycidyl ether, 1,3-bis(glycidoxypropyl)tetramethyldisiloxane, methoxymethylated urea, butoxymethylated urea, methoxymethylated thiourea, and/or the like.

The leveling agent may be used for improving coating flatness during printing and may be a commercially available suitable leveling agent.

The organic acid may include p-toluenesulfonic acid, benzenesulfonic acid, p-dodecylbenzenesulfonic acid, 1,4-naphthalenedisulfonic acid, methanesulfonic acid, a fluorinated sulfonium salt, malonic acid, citric acid, propionic acid, methacrylic acid, oxalic acid, lactic acid, glycolic acid, succinic acid, and/or a (e.g., any suitable) combination thereof, but embodiments of the present disclosure are not limited thereto.

The quencher may be diphenyl (p-tolyl) amine, methyl diphenyl amine, triphenyl amine, phenylenediamine, naphthylamine, diaminonaphthalene, or a (e.g., any suitable) combination thereof.

A amount of the additives included in the semiconductor photoresist composition may be controlled or selected depending on desired or suitable properties.

In one or more embodiments, the semiconductor photoresist composition may further include a silane coupling agent as an adherence enhancer in order to improve a close-contacting force with the substrate (e.g., in order to improve adherence of the semiconductor photoresist composition to the substrate). The silane coupling agent may be, for example, a silane compound including a carbon-carbon unsaturated bond such as vinyltrimethoxysilane, vinyl triethoxysilane, vinyl trichlorosilane, vinyl tris(β-methoxyethoxy) silane, or 3-methacryloxypropyltrimethoxysilane, 3-acryloxypropyltrimethoxysilane, p-styryl trimethoxysilane, 3-methacryloxypropylmethyldimethoxysilane, 3-methacryloxypropylmethyl diethoxysilane; trimethoxy [3-(phenylamino) propyl]silane; and/or the like, but embodiments of the present disclosure are not limited thereto.

The semiconductor photoresist composition may be formed into a pattern having a high aspect ratio without a collapse. Accordingly, in order to form a fine pattern having a width (e.g., line width) of, for example, about 5 nm to about 100 nm, for example, about 5 nm to about 80 nm, for example, about 5 nm to about 70 nm, for example, about 5 nm to about 50 nm, for example, about 5 nm to about 40 nm, for example, about 5 nm to about 30 nm, or for example, about 5 nm to about 20 nm, the semiconductor photoresist composition may be used for a photoresist process using light in a wavelength in a range of about 5 nm to about 150 nm, for example, about 5 nm to about 100 nm, about 5 nm to about 80 nm, about 5 nm to about 50 nm, about 5 nm to about 30 nm, or about 5 nm to about 20 nm. Accordingly, the semiconductor photoresist composition according to one or more embodiments may be used to realize extreme ultraviolet lithography using an EUV light source of a wavelength of about 13.5 nm.

According to one or more embodiments, a method of forming patterns using the aforementioned semiconductor photoresist composition is provided. For example, the manufactured pattern may be a photoresist pattern.

The method of forming patterns according to one or more embodiments includes forming an etching-objective layer (e.g., etching-target layer) on a substrate, coating the semiconductor photoresist composition on the etching-objective layer to form a photoresist layer, patterning the photoresist layer to form a photoresist pattern, and etching the etching-objective layer using the photoresist pattern as an etching mask.

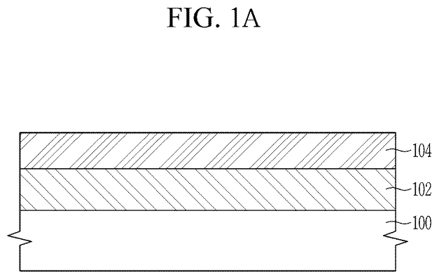

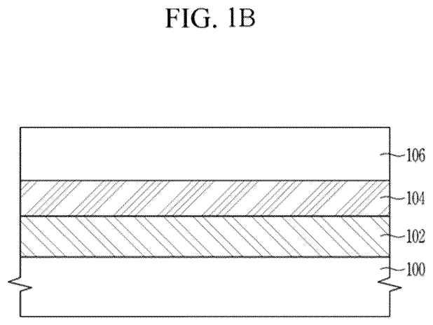

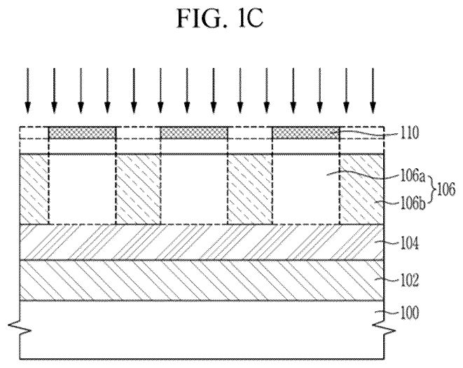

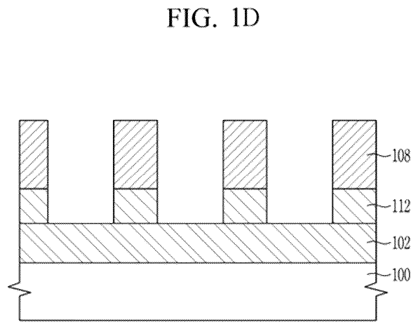

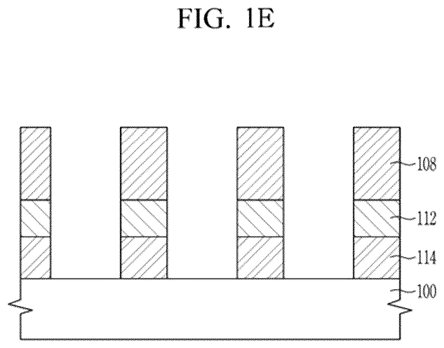

Hereinafter, a method of forming patterns using the semiconductor photoresist composition will be described in more detail by referring to FIGS. 1A-1E. FIGS. 1A-1E are cross-sectional views for illustrating a method of forming patterns using a semiconductor photoresist composition according to one or more embodiments.

Referring to FIG. 1A, an object for etching (e.g., etching-objective layer or etching-target layer) is prepared. The object for etching may be a thin film 102 formed on a semiconductor substrate 100. Hereinafter, the object for etching is limited to the thin film 102. A surface of the thin film 102 is washed to remove impurities and/or the like remaining thereon. The thin film 102 may be, for example, a silicon nitride layer, a polysilicon layer, or a silicon oxide layer.

Subsequently, a resist underlayer composition for forming a resist underlayer 104 is spin-coated on the surface of the washed thin film 102. However, embodiments of the present disclosure are not limited thereto, and one or more suitable coating methods, for example, a spray coating, a dip coating, a knife edge coating, a printing method (for example, an inkjet printing and/or a screen printing), and/or the like, may be used.

In one or more embodiments, the coating process of the resist underlayer may not be provided, and hereinafter, a process including a coating of the resist underlayer is described.

Then, the coated resist underlayer composition is dried and baked to form the resist underlayer 104 on the thin film 102. The baking (e.g., thermal treatment) may be performed at about 100° C. to about 500° C., for example, about 100° C. to about 300° C.

The resist underlayer 104 is formed between the substrate 100 and a photoresist layer 106 and thus may prevent or reduce non-uniformity and pattern formability of a photoresist line width if (e.g., when) a ray reflected from on the interface between the substrate 100 and the photoresist layer 106 or a hardmask between layers is scattered into an unintended photoresist region.

Referring to FIG. 1B, the photoresist layer 106 is formed by coating the semiconductor photoresist composition on the resist underlayer 104. The photoresist layer 106 is obtained by coating the aforementioned semiconductor photoresist composition on the thin film 102 formed on the substrate 100 and then, curing it through a heat treatment.

In one or more embodiments, the formation of a pattern by using the semiconductor photoresist composition may include coating the semiconductor resist composition on the substrate 100 having the thin film 102 through spin coating, slit coating, inkjet printing, and/or the like and then, drying it to form the photoresist layer 106.

The semiconductor photoresist composition has already been illustrated in detail and will not be illustrated again.

Subsequently, the substrate 100 having the photoresist layer 106 is subjected to a first baking (e.g., thermal treatment) process. The first baking process may be performed at about 80° C. to about 120° C.

Referring to FIG. 1C, the photoresist layer 106 may be selectively exposed using a patterned mask 110.

For example, the exposure may use an activation radiation with light having a high energy wavelength such as EUV (extreme ultraviolet; a wavelength of about 13.5 nm), an E-Beam (an electron beam), and/or the like as well as a short wavelength such as an i-line (a wavelength of about 365 nm), a KrF excimer laser (a wavelength of about 248 nm), an ArF excimer laser (a wavelength of about 193 nm), and/or the like.

For example, light or exposure beam for the exposure according to one or more embodiments may have a short wavelength in a range of about 5 nm to about 150 nm, and/or a high energy wavelength, for example, may be an EUV (extreme ultraviolet; a wavelength of 13.5 nm), and/or may be an E-Beam (an electron beam), and/or the like.

The exposed region 106b of the photoresist layer 106 has a different solubility from the unexposed region 106a of the photoresist layer 106 by forming a polymer by a crosslinking reaction such as condensation between organometallic compounds.

Subsequently, the substrate 100 is subjected to a second baking (thermal treatment) process. The second baking process may be performed at a temperature of about 90° C. to about 200° C. The exposed region 106b of the photoresist layer 106 becomes indissoluble regarding a developer due to the second baking process.

In FIG. 1D, the unexposed region 106a of the photoresist layer is dissolved and removed using the developer to form a photoresist pattern 108. For example, the unexposed region 106a of the photoresist layer is dissolved and removed by using an organic solvent such as 2-heptanone and/or the like to complete the photoresist pattern 108 corresponding to a negative tone image.

As described above, a developer used in the method of forming patterns according to one or more embodiments may be an organic solvent. The organic solvent used in the method of forming patterns according to one or more embodiments may be, for example, a ketone such as methylethylketone, acetone, cyclohexanone, 2-heptanone, and/or the like, an alcohol such as 4-methyl-2-propanol, 1-butanol, isopropanol, 1-propanol, methanol, and/or the like, an ester such as propylene glycol monomethyl ether acetate, ethyl acetate, ethyl lactate, n-butyl acetate, butyrolactone, and/or the like, an aromatic compound such as benzene, xylene, toluene, and/or the like, or a (e.g., any suitable) combination thereof.

However, the photoresist pattern according to one or more embodiments is not necessarily limited to the negative tone image but may be formed to have a positive tone image. Herein, a developer used for forming the positive tone image may be a quaternary ammonium hydroxide composition such as tetraethylammonium hydroxide, tetrapropylammonium hydroxide, tetrabutylammonium hydroxide, or a (e.g., any suitable) combination thereof.

As described above, exposure to light having a high energy such as EUV (extreme ultraViolet; a wavelength of 13.5 nm), to an E-Beam (an electron beam), and/or the like, and/or to light having a short wavelength such as i-line (wavelength of about 365 nm), KrF excimer laser (wavelength of about 248 nm), ArF excimer laser (wavelength of about 193 nm), and/or the like may provide a photoresist pattern 108 having a width of a thickness of about 5 nm to about 100 nm. For example, in one or more embodiments, the photoresist pattern 108 may have a width of a thickness of about 5 nm to about 90 nm, about 5 nm to about 80 nm, about 5 nm to about 70 nm, about 5 nm to about 60 nm, about 5 nm to about 50 nm, about 5 nm to about 40 nm, about 5 nm to about 30 nm, or about 5 nm to about 20 nm.

In one or more embodiments, the photoresist pattern 108 may have a pitch (center-to-center distance between adjacent features in the pattern) having (with) a half-pitch of less than or equal to about 50 nm, for example less than or equal to about 40 nm, for example less than or equal to about 30 nm, for example less than or equal to about 20 nm, or for example less than or equal to about 15 nm, and a line width roughness of less than or equal to about 10 nm, or less than or equal to about 5 nm, less than or equal to about 3 nm, or less than or equal to about 2 nm.

Subsequently, the photoresist pattern 108 is used as an etching mask to etch the resist underlayer 104. Through this etching process, an organic layer pattern 112 is formed. The organic layer pattern 112 also may have a width corresponding to that of the photoresist pattern 108.

Referring to FIG. 1E, the exposed thin film 102 is etched by applying the photoresist pattern 108 as an etching mask. As a result, the thin film is formed as a thin film pattern 114.

The etching of the thin film 102 may be, for example, dry etching using an etching gas, and the etching gas may be, for example, CHF₃, CF₄, Cl₂, BCl₃, or a mixed gas thereof.

In the exposure process, the thin film pattern 114 formed by using the photoresist pattern 108 formed through the exposure process performed by using an EUV light source may have a width corresponding to that of the photoresist pattern 108. For example, in one or more embodiments, the thin film pattern 114 may have a width (e.g., line width) of about 5 nm to about 100 nm which is equal to that of the photoresist pattern 108. For example, in one or more embodiments, the thin film pattern 114 formed by using the photoresist pattern 108 formed through the exposure process performed by using an EUV light source may have a width (e.g., line width) of about 5 nm to about 90 nm, about 5 nm to about 80 nm, about 5 nm to about 70 nm, about 5 nm to about 60 nm, about 5 nm to about 50 nm, about 5 nm to about 40 nm, about 5 nm to about 30 nm, or about 5 nm to about 20 nm, for example, a width (e.g., line width) of less than or equal to about 20 nm, like that of the photoresist pattern 108.

Hereinafter, the present disclosure will be described in more detail through examples of the preparation of the aforementioned semiconductor photoresist composition. However, the present disclosure is technically not restricted by the following examples.

### Synthesis of Organometallic Compounds

### Synthesis Example 1

40.7 g of t-butylSnPhs and 300 g of propionic acid were added to a 250 ml 2-necked round-bottomed flask and then, refluxed by heating for 24 hours.

A compound represented by Chemical Formula 6 was obtained by removing the unreacted propionic acid under a reduced pressure therefrom.

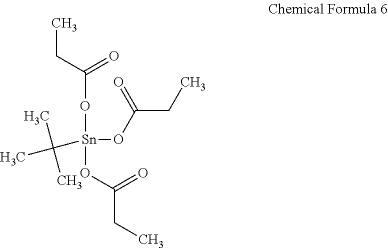 [SMILES: CCC(=O)[O][Sn]([O]C(=O)CC)([O]C(=O)CC)[C](C)(C)C]

### Synthesis Example 2

After adding 30 mL of anhydrous pentane to 10 g of t-AmylSnCl₃ and maintaining their temperature at 0° C., 7.4 g of diethyl amine and 6.1 g of ethanol were added thereto and then, stirred at room temperature for 1 hour. When a reaction was completed, the resultant was filtered, concentrated, and vacuum-dried, obtaining a compound represented by Chemical Formula 7.

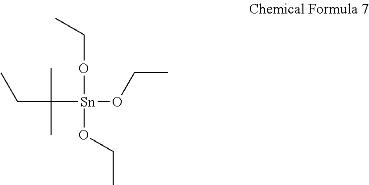

### Synthesis Example 3

10 g of dibutyltin dichloride was dissolved 30 mL of ether, 70 mL of a 1 M sodium hydroxide (NaOH) aqueous solution was added thereto and then, stirred for 1 hour. After the stirring, a solid produced therein was filtered, washed with 25 mL of deionized water for three times, and dried at 100° C. under a reduced pressure to obtain an organometallic compound represented by Chemical Formula 8 and having a weight average molecular weight of 1,500.

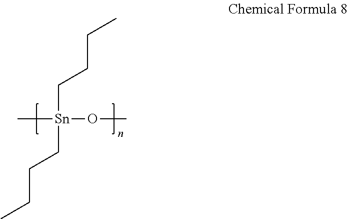

### Preparation of Semiconductor Photoresist Compositions

### Examples 1 to 11 and Comparative Examples 1 to 3

According to Table 1, respective Sn-containing organometallic compound selected from among Sn-containing organometallic compounds represented by Chemical Formulas 6 to 8 obtained in Synthesis Examples 1 to 3 and respective carboxylic acid compound were dissolved in Propylene glycol methyl ether acetate (PGMEA) at a concentration of 3 wt % at a weight ratio shown in Table 1, and then, filtered with a 0.1 μm PTFE (polytetrafluoroethylene) syringe filter to prepare semiconductor photoresist compositions according to Examples 1 to 11 and Comparative Examples 1 to 3.

TABLE 1

| | Organometallic | Carboxylic acid |
|---|---|---|
| | compound | compound |
| | (wt %) | (wt %) |
| Example 1 | Chemical Formula 6 | Cinnamic acid |
| | (2.5) | (0.5) |
| Example 2 | Chemical Formula 6 | 3-Methylcinnamic acid |
| | (2.5) | (0.5) |
| Example 3 | Chemical Formula 6 | 2,3-Dimethylcinnamic |
| | (2.5) | acid |
| | | (0.5) |
| Example 4 | Chemical Formula 6 | Cinnamic acid |
| | (2.95) | (0.05) |
| Example 5 | Chemical Formula 6 | Cinnamic acid |
| | (2.80) | (0.20) |
| Example 6 | Chemical Formula 7 | Cinnamic acid |
| | (2.85) | (0.15) |
| Example 7 | Chemical Formula 7 | 3-Methylcinnamic acid |
| | (2.85) | (0.15) |
| Example 8 | Chemical Formula 7 | 2,3-Dimethylcinnamic |
| | (2.85) | acid |
| | | (0.15) |
| Example 9 | Chemical Formula 8 | Cinnamic acid |
| | (2.85) | (0.15) |
| Example 10 | Chemical Formula 8 | 3-Methylcinnamic acid |
| | (2.85) | (0.15) |
| Example 11 | Chemical Formula 8 | 2,3-Dimethylcinnamic |
| | (2.85) | acid |
| | | (0.15) |
| Comparative Example 1 | Chemical Formula 6 | — |
| | 3.00 | |
| Comparative Example 2 | Chemical Formula 6 | crotonic acid |
| | (2.85) | (0.15) |
| Comparative Example 3 | Chemical Formula 6 | benzoic acid |
| | (2.85) | (0.15) |

### Evaluation 1: Evaluation of Sensitivity and Line Edge Roughness (LER)

Each of the photoresist compositions according to the examples and the comparative examples was spin-coated at 1500 rpm for 30 seconds on a 200 mm circular silicon wafer of which the surface was deposited with hexamethyldisilazane (HMDS), baked at 110° C. for 60 seconds (post-apply baked (PAB)), and then, allowed to stand at room temperature (23+2° C.) for 30 seconds.

Subsequently, a linear array of 50 circular pads with a diameter of 500 μm was projected onto the wafer coated with one of the photoresist compositions by using EUV light (Lawrence Berkeley National Laboratory Micro Exposure Tool, MET). Herein, pad exposure time was adjusted to ensure that the EUV light in an increased dose was applied to each pad.

Then, the resist and the substrate were baked at 160° C. for 120 seconds on a hot plate after the exposure. The baked film was developed in a PGMEA solvent to form a negative tone image. Finally, the obtained film was baked again at 150° C. for 2 minutes on the hot plate, completing the process.

Critical dimension scanning electron microscope (CD-SEM) was used to measure resist line widths to exposed dose (energy) changes. Appropriate sensitivity to the exposure amount was confirmed from the resist line width values formed differently depending on each exposure dose. The resolution was confirmed by measuring the resist line width formed by exposing the full wafer at the same dose with the confirmed appropriate or suitable sensitivity. In addition, after measuring line edge roughness (LER) from the CD-SEM image, sensitivity and LER were evaluated according to the following criteria, and the results are shown in Table 2.

### Evaluation Criteria of Sensitivity

A: less than 16 mJ/cm²
B: greater than or equal to 16 mJ/cm² and less than 18 mJ/cm²
C: greater than or equal to 18 mJ/cm²

### Evaluation Criteria of LER

∘: less than 2 nm
Δ: greater than or equal to 2 nm and less than 5 nm
X: greater than or equal to 5 nm

### Resolution Criteria

A: less than 14.3
B: greater than or equal to 14.3 and less than 15.2
C: greater than or equal to 15.2

TABLE 2

| | | | Resolution |
|---|---|---|---|
| | | | minimum |
| | | | space CD |
| | Sensitivity | LER | (@14 nm) |
| Example 1 | A | ◯ | A |
| Example 2 | A | ◯ | A |
| Example 3 | A | ◯ | A |
| Example 4 | B | ◯ | A |
| Example 5 | A | ◯ | B |
| Example 6 | A | ◯ | B |
| Example 7 | B | ◯ | A |
| Example 8 | A | Δ | B |
| Example 9 | B | Δ | A |
| Example 10 | A | Δ | B |
| Example 11 | A | Δ | B |
| Comparative Example 1 | C | X | C |
| Comparative Example 2 | C | Δ | C |
| Comparative Example 3 | B | X | C |

Referring to the results of Table 2, the patterns formed by respectively using the semiconductor photoresist compositions according to Examples 1 to 11 exhibited excellent or suitable sensitivity and LER and/or EL characteristics, compared with those formed by respectively using the semiconductor photoresist compositions according to Comparative Examples 1 to 3.

As utilized herein, the terms “and/or” and “or” may include any and all combinations of one or more of the associated listed items. Expressions such as “at least one of,” when preceding a list of elements, modify the entire list of elements and do not modify the individual elements of the list.

It will be further understood that the terms “comprise”, “include,” or “have/has,” when utilized in the present disclosure, specify the presence of stated features, integers, steps, operations, elements, and/or components, but do not preclude the presence or addition of one or more other features, integers, steps, operations, elements, components, and/or groups thereof. The “/” utilized below may be interpreted as “and” or as “or” depending on the situation.

As utilized herein, the singular forms “a,” “an,” and “the” are intended to include the plural forms as well, unless the context clearly indicates otherwise. Further, the utilization of “may” when describing embodiments of the present disclosure refers to “one or more embodiments of the present disclosure”.

In the context of the present disclosure and unless otherwise defined, the terms “use,” “using,” and “used” may be considered synonymous with the terms “utilize,” “utilizing,” and “utilized,” respectively.

As utilized herein, the term “about,” or similar terms are used as terms of approximation and not as terms of degree, and are intended to account for the inherent deviations in measured or calculated values that would be recognized by those of ordinary skill in the art. “About” or “approximately,” as used herein, is also inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within +30%, 20%, 10%, or 5% of the stated value.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

A person of ordinary skill in the art would appreciate, in view of the present disclosure in its entirety, that each suitable feature of the various embodiments of the present disclosure may be combined or combined with each other, partially or entirely, and may be technically interlocked and operated in various suitable ways, and each embodiment may be implemented independently of each other or in conjunction with each other in any suitable manner unless otherwise stated or implied.

A pattern forming device, a semiconductor forming device and/or any other relevant devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the present disclosure.

Hereinbefore, the example embodiments have been described and illustrated, however, it is apparent to a person with ordinary skill in the art that the present disclosure is not limited to the example embodiments as described, and may be variously modified and transformed without departing from the spirit and scope of the present disclosure. Accordingly, the modified or transformed embodiments as such may not be understood separately from the technical ideas and aspects of the present disclosure, and the modified embodiments are within the scope of the claims and equivalents thereof, of the present disclosure.

Reference Numerals

| | |
|---|---|
| 100: substrate | 102: thin film |
| 104: resist underlayer | 106: photoresist layer |
| 106a: unexposed region | 106b: exposed region |
| 108: photoresist pattern | 112: organic layer pattern |
| 110: patterned mask | 114: thin film pattern |

What is claimed is:

1. A semiconductor photoresist composition, comprising:
a Sn-containing organometallic compound;
a carboxylic acid compound comprising at least one aryl group and an unsaturated bond; and
a solvent.

2. The semiconductor photoresist composition as claimed in claim 1, wherein
the carboxylic acid compound is represented by Chemical Formula 1 or Chemical Formula 2: 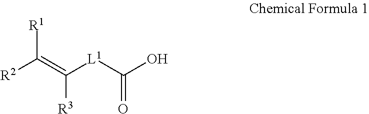
in Chemical Formula 1,
R¹ to R³ being each independently hydrogen, a halogen, a hydroxyl group, a substituted or unsubstituted C1 to C20 alkyl group, a substituted or unsubstituted C6 to C30 aryl group, or a combination thereof,
at least one of selected from among R¹ to R³ being a substituted or unsubstituted C6 to C30 aryl group, and
L¹ being a single bond or a substituted or unsubstituted C1 to C10 alkylene group; and 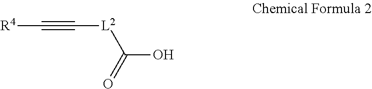
in Chemical Formula 2,
R⁴ being a substituted or unsubstituted C6 to C30 aryl group, and
L² being a single bond or a substituted or unsubstituted C1 to C10 alkylene group.

3. The semiconductor photoresist composition as claimed in claim 2, wherein
at least one selected from among R¹ and R² or the R³ is each independently a substituted or unsubstituted C6 to C30 aryl group.

4. The semiconductor photoresist composition as claimed in claim 2, wherein
at least one selected from among R¹ and R² or the R³, and the R⁴ are each independently a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, or a substituted or unsubstituted triphenylene group.

5. The semiconductor photoresist composition as claimed in claim 1, wherein
the carboxylic acid compound is any one selected from among compounds listed in Group 1:

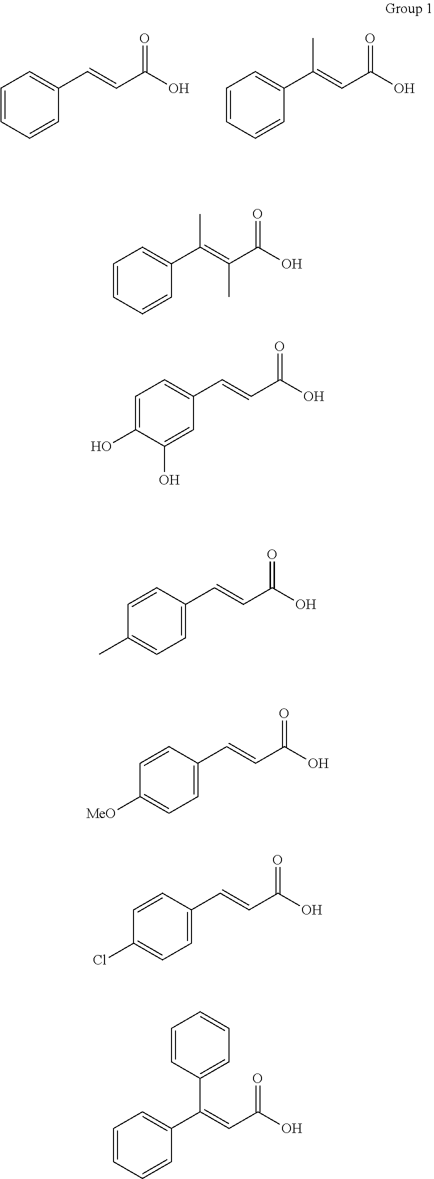

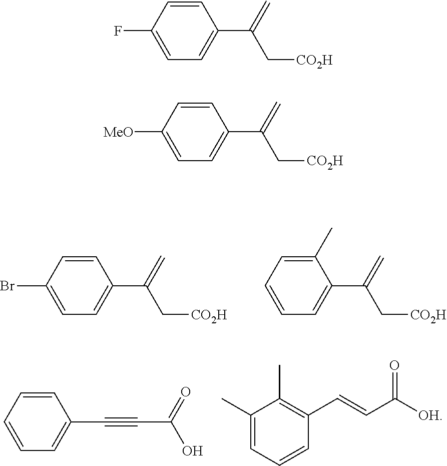

6. The semiconductor photoresist composition as claimed in claim 1, wherein
the carboxylic acid compound is in an amount of about 0.01 to about 10 wt % based on 100 wt % of a total weight of the semiconductor photoresist composition.

7. The semiconductor photoresist composition as claimed in claim 1, wherein
the carboxylic acid compound is in an amount of about 0.5 wt % to about 5 wt % based on 100 wt % of a total weight of the semiconductor photoresist composition.

8. The semiconductor photoresist composition as claimed in claim 1, wherein
the Sn-containing organometallic compound is in an amount of about 0.5 wt % to about 30 wt % based on 100 wt % of a total weight of the semiconductor photoresist composition.

9. The semiconductor photoresist composition as claimed in claim 1, wherein
the semiconductor photoresist composition further comprises an additive selected from among a surfactant, a crosslinking agent, a leveling agent, an organic acid, a quencher, and combinations thereof.

10. The semiconductor photoresist composition as claimed in claim 1, wherein
the Sn-containing organometallic compound comprises at least one of an organic oxy group or an organic carbonyloxy group.

11. The semiconductor photoresist composition as claimed in claim 1, wherein
the Sn-containing organometallic compound is represented by Chemical Formula 3: 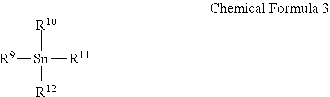
in Chemical Formula 3,
R⁹ being selected from among a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, and a substituted or unsubstituted C7 to C30 arylalkyl group,
R¹⁰ to R¹² being each independently a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, a substituted or unsubstituted C7 to C30 arylalkyl group, an alkoxy or aryloxy group (—OR^{a}, wherein R^{a} is a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), a carboxyl group (—O(C═O)R^{b}, wherein R^{b} is hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), an alkylamido or dialkylamido group (—NR^{c}R^{d}, wherein R^{c} and R^{d} are each independently hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), an amidato group (—NR^{e}(C═OR^{f}), wherein R^{e} and R^{f} are each independently hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), an amidinato group (—NR^{g}C(NR^{h})Rⁱ, wherein R^{g}, R^{h}, and Rⁱ are each independently hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), an alkylthio or arylthiol group (—SRⁱ, wherein Rⁱ is a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), or a thiocarboxyl group (—S(C═O)R^{k}, wherein R^{k} is hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), and
at least one selected from among R¹⁰ to R¹² being selected from among an alkoxy or aryloxy group (—OR^{a}, wherein R^{a} is a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), a carboxyl group (—O(C═O)R^{b}, wherein R^{b} is hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), an alkylamido or dialkylamido group (—NR^{c}R^{d}, wherein R^{c} and R^{d} are each independently hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), an amidato group (—NR^{e}(C═OR^{f}), wherein R^{e} and R^{f} are each independently hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), an amidinato group (—NR^{g}C(NR^{h})Rⁱ, wherein R^{g}, R^{h}, and Rⁱ are each independently hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), an alkylthio or arylthiol group (—SRⁱ, wherein Rⁱ is a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), and a thiocarboxyl group (—S(C═O)R^{k}, wherein R^{k} is hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof).

12. The semiconductor photoresist composition as claimed in claim 11, wherein
at least one selected from among R¹⁰ to R¹² is selected from among an alkoxy or aryloxy group (—OR^{a}, wherein R^{a} is a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), and a carboxyl group (—O(C═O)R^{b}, wherein R^{b} is hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof).

13. The semiconductor photoresist composition as claimed in claim 11, wherein
R⁹ is a substituted or unsubstituted C1 to C8 alkyl group, a substituted or unsubstituted C3 to C8 cycloalkyl group, a substituted or unsubstituted C2 to C8 aliphatic unsaturated organic group comprising one or more double bonds or triple bonds, a substituted or unsubstituted C6 to C20 aryl group, a substituted or unsubstituted C4 to C20 heteroaryl group, a carbonyl group, an ethoxy group, a propoxy group, or a combination thereof,
R^{a} is a substituted or unsubstituted C1 to C8 alkyl group, a substituted or unsubstituted C3 to C8 cycloalkyl group, a substituted or unsubstituted C2 to C8 alkenyl group, a substituted or unsubstituted C2 to C8 alkynyl group, a substituted or unsubstituted C6 to C20 aryl group, or a combination thereof, and
R^{b} is hydrogen, a substituted or unsubstituted C1 to C8 alkyl group, a substituted or unsubstituted C3 to C8 cycloalkyl group, a substituted or unsubstituted C2 to C8 alkenyl group, a substituted or unsubstituted C2 to C8 alkynyl group, a substituted or unsubstituted C6 to C20 aryl group, or a combination thereof.

14. The semiconductor photoresist composition as claimed in claim 1, wherein
the Sn-containing organometallic compound is represented by Chemical Formula 4 or Chemical Formula 5:
R¹³_{z}SnO_{(2-(z/2)-(x/2))}(OH)ₓ Chemical Formula 4
in Chemical Formula 4,
R¹³ being a C1 to C31 hydrocarbyl group, 0<z≤2, and 0<(z+x)≤4; and
R¹⁴ₐ₁Sn_{b1}X_{c1}Y_{d1} Chemical Formula 5
in Chemical Formula 5,
R¹⁴ being a substituted or unsubstituted C1 to C20 alkyl group, a substituted or unsubstituted C3 to C20 cycloalkyl group, a substituted or unsubstituted C2 to C20 aliphatic unsaturated organic group comprising one or more double bonds or triple bonds, a substituted or unsubstituted C6 to C30 aryl group, a substituted or unsubstituted C4 to C30 heteroaryl group, a carbonyl group, an ethylene oxide group, a propylene oxide group, or a combination thereof,
X being sulfur(S), selenium (Se), or tellurium (Te),
Y being-ORI or OC(═O)R^{m},
wherein R^{l} is a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof,
R^{m} is hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof, and
a1, b1, c1, and d1 being each independently an integer of 1 to 20.

15. A method of forming patterns, comprising:
forming an etching-objective layer on a substrate;
coating the semiconductor photoresist composition as claimed in claim 1 on the etching-objective layer to form a photoresist layer;
patterning the photoresist layer to form a photoresist pattern; and
etching the etching-objective layer utilizing the photoresist pattern as an etching mask.
